(19) United States
(12) Patent Application Publication
(10) Pub. No.: US 2015/0307411 A1
(43) Pub. Date: Oct. 29, 2015

(54) PROCESS FOR PREPARING ETHYLENE AND/OR PROPYLENE

(71) Applicants: SHELL INTERNATIONALE RESEARCH MAATSCHAPPIJ B.V., The Hague (NL); SHELL OIL COMPANY, Houston, TX (US)

(72) Inventors: Ye Mon CHEN, Sugar Land, TX (US); Timothy Michael NISBET, Amsterdam (NL); Sivakumar SADASIVAN VIJAYAKUMARI, Amsterdam (NL); Jeroen WESTRENEN, Amsterdam (NL)

(21) Appl. No.: 14/368,547

(22) PCT Filed: Dec. 21, 2012
(86) PCT No.: PCT/EP2012/076847

§ 371 Date: Jun. 25, 2014

Related U.S. Application Data
(60) Provisional application No. 61/580,383, filed on Dec. 27, 2011.

(51) Int. Cl. C07C 1/20; C07C 4/06

(52) U.S. Cl. CPC C07C 1/20; C07C 4/06; C07C 2529/40

(57) ABSTRACT

The present invention provides a process for preparing ethylene and/or propylene, comprising the steps of providing an oxygenate feed comprising oxygenate and C4 olefins to a first reaction zone; contacting the oxygenate feed with a first zeolite-comprising catalyst and retrieving from the first reaction zone a first effluent stream comprising at least C2 to C5 olefins; separating the first effluent stream into at least: a first product stream comprising C2 and/or C3 olefins; a second fraction comprising C4 olefins; a third fraction comprising C5 olefins; and recycling at least part of the second fraction to the first reaction zone as part of the oxygenate feed; providing an olefinic feed comprising C5 olefins to a second reaction zone, wherein the olefinic feed comprises at least part of the third fraction; and contacting in the second reaction zone the olefinic feed with a second zeolite-comprising catalyst at a temperature in the range of from 500 to 700° C. and retrieving from the second reaction zone a second effluent stream comprising at least C2 to C3 olefins.

### FIELD OF THE INVENTION

This invention relates to a process for preparing ethylene and/or propylene.

### BACKGROUND TO THE INVENTION

Conventionally, ethylene and propylene are produced via steam cracking of paraffinic feedstocks including ethane, propane, naphtha and hydrowax. An alternative route to ethylene and propylene is an oxygenate-to-olefin (OTO) process. Interest in OTO processes for producing ethylene and propylene is growing in view of the increasing availability of natural gas. Methane in the natural gas can be converted, for instance, to methanol or dimethylether (DME), both of which are suitable feedstocks for an OTO process.

In an OTO process, an oxygenate such as methanol is provided to a reaction zone comprising a suitable conversion catalyst and converted to ethylene and propylene. In addition to the desired ethylene and propylene, a substantial part of the methanol is converted to higher hydrocarbons including C4+ olefins.

These C4+ olefins may be recycled and provided together with the oxygenate to the OTO reaction zone. Such a process is for instance described in U.S. Pat. No. 6,441,261, wherein it is mentioned that C4+ hydrocarbon mixtures that are obtained from separation and recycle of the reaction product are co-fed to the reactor together with the oxygenate.

In WO2009/156433, an alternative is proposed to recycling the C4+ fraction in the reaction product to the reaction zone to be co-fed together with the oxygenate.

In order to increase the ethylene and propylene yield of the process, WO2009/156433 proposes to further crack the C4+ olefins in a dedicated olefin cracking zone to produce further ethylene and propylene. In WO2009/156433, a process is described, wherein an oxygenate feedstock is converted in an OTO zone (XTO zone) to an ethylene and propylene product. Higher olefins, i.e. C4+ olefins, produced in the OTO zone are directed to an olefin cracking zone (OC zone). In the olefin cracking zone, part of the higher olefins is converted to additional ethylene and propylene to increase the overall selectivity of the process to ethylene and propylene.

A disadvantage of the process of WO2009/156433 is that it requires an additional olefin cracking zone, with accompanying CAPEX, while the increase in the yield of ethylene and propylene of the overall process is minimal as the C4+ olefins cracking is not particularly selective for ethylene and propylene. The observed yield of ethylene and propylene was increased by a mere 0.4wt %. This disadvantage becomes even more pronounced should it be desired to additionally feed external C4+ olefins, together with the C4+ olefins obtained from the OTO zone, to the olefin cracking zone.

There is a need in the art for a process for producing ethylene and propylene from an oxygenate feed, wherein the yield of and selectivity to ethylene and propylene is increased, even in case an external feed comprising C4+ olefins, in particular comprising C4 and C5+ olefins, is provided to the process in addition to the oxygenate feed.

### SUMMARY OF THE INVENTION

It has now been found that an increased yield of and selectivity to ethylene and propylene can be achieved when converting oxygenates to olefins in a process wherein C4 olefins and C5+ olefins, present in the reaction effluent of an oxygenate-to-olefins reaction zone, are separately further converted to ethylene and propylene.

Accordingly, the present invention provides a process for preparing ethylene and/or propylene, comprising the steps of: a) providing an oxygenate feed comprising oxygenate and C4 olefins to a first reaction zone; b) contacting in the first reaction zone the oxygenate feed with a first zeolite-comprising catalyst at a temperature in the range of from 350 to 1000° C. and retrieving from the first reaction zone a first effluent stream comprising at least C2 to C5 olefins; c) separating the first effluent stream into at least: a first product stream comprising C2 and/or C3 olefins; a second fraction comprising C4 olefins; and a third fraction comprising C5 olefins; d) recycling at least part of the second fraction to the first reaction zone as part of the oxygenate feed; e) providing an olefinic feed comprising C5 olefins to a second reaction zone, wherein the olefinic feed comprises at least part of the third fraction; and f) contacting in the second reaction zone the olefinic feed with a second zeolite-comprising catalyst at a temperature in the range of from 500 to 700° C. and retrieving from the second reaction zone a second effluent stream comprising at least C2 to C3 olefins.

By converting the C5 olefins separately from the C4 olefins a higher yield of ethylene and propylene can be achieved. C4 olefins are more difficult the crack to ethylene and propylene and therefore require more severe cracking conditions. By recycling the C4 olefins and converting the C4 olefins in the presence of an oxygenate, such as methanol or DME, rather than cracking them together with the C5 olefins, a higher total yield of ethylene and propylene can be achieved.

In addition, the process according to the present invention allows for preparing ethylene and/or propylene from oxygenates with a lowered by-product make in particular a lower paraffins and aromatics make.

### DETAILED DESCRIPTION OF THE INVENTION

Ethylene and/or propylene can be produced from oxygenates such as methanol and dimethylether (DME) through an oxygenate-to-olefins (OTO) process. Such processes are well known in the art and are also referred to as methanol-to-olefins or methanol-to-propylene processes. In an OTO process, typically the oxygenate is contacted with a zeolite-comprising catalyst at elevated temperatures. In contact with the zeolite-comprising catalyst, the oxygenate is converted into ethylene and/or propylene. Besides ethylene and propylene, substantial amounts of C4+ olefins are produced. To increase the total yield of ethylene and propylene, these C4+ olefins may be converted to obtain further ethylene and propylene. One way of converting the C4+ olefins to ethylene and propylene is through cracking the C4+ olefins by contacting the C4+ olefins at elevated temperature with a zeolite-comprising catalyst. This process is generally referred to as an olefin cracking process or OCP.

In the process according to the present invention, rather than sending the C4+ olefins as a single feed to the OCP, the C4+ olefins obtained from the OTO process are separated into at least a fraction comprising C4 olefins and a fraction comprising C5 olefins. The fraction comprising C4 olefins is recycled back to the OTO process to be contacted with the zeolite catalyst together with the oxygenate, while the fraction comprising C5 olefins is sent to the OCP.

Without wishing to be bound by any particular theory, it is believed that the cracking behaviour of C4 olefins and C5 olefins, when contacted with a zeolite-comprising catalyst, is different, in particular above 500° C. The cracking of C4 olefins is an indirect process which involves a primary oligomerisation process to a C8, C12 or higher olefin followed by cracking of the oligomers to lower molecular weight hydrocarbons including ethylene and propylene, but also, amongst other things, to C5 to C7 olefins, and by-products such as C2 to C6 paraffins, cyclic and aromatics. In addition, the cracking of C4 olefins is prone to coke formation, which places a restriction on the desired conversion of the C4 olefins. Generally, paraffins, cyclics and aromatics are not formed by cracking. They are formed by hydrogen transfer reactions, optionally, followed by cyclisation. This is more likely in larger molecules. Hence the C4 olefin cracking process, which as mentioned above includes intermediate oligomerisation, is more prone to by-product formation than direct cracking of C5 olefins. The conversion of the C4 olefins is typically a function of the temperature and space time (often expressed as the weight hourly space velocity, [kg_{c4-feed}/(kg_{catalyst}.hr)]). With increasing temperature and decreasing weight hourly space velocity (WHSV) conversion of the C4 olefins in the feed to the OCP increases. Initially, the ethylene and propylene yields increase, but, at higher conversions, yield decreases at the cost of a higher by-product make and, in particular, a higher coke make, limiting significantly the maximum yield obtainable.

Contrary to C4 olefins, C5 olefin cracking is ideally a relatively straight forward-process whereby the C5 olefin cracks into a C2 and a C3 olefin, in particular above 500° C. This cracking reaction can be run at high conversions, up to 100%, while maintaining, at least compared to C4 olefins, high ethylene and propylene yields with a significantly lower by-product and coke make. Although, C5+ olefins can also oligomerise, this process competes with the more beneficial cracking to ethylene and propylene.

In the process according to the present invention, instead of cracking the C4 olefins in the OCP, the C4 olefins are recycled to the OTO reaction. Again without wishing to be bound by any particular theory, it is believed that in the OTO reaction the C4 olefins are alkylated with, for instance, methanol to C5 and/or C6 olefins. These C5 and/or C6 olefins may subsequently be converted into at least ethylene and/or propylene. The main by-products from this OTO reaction are again C4 and C5 olefins, which can be recycled to the OTO reactor and OCP respectively.

The process according to the invention is now described in more detail herein below.

The process according to the present invention is a process for preparing ethylene and/or propylene. In the process according to the invention, an oxygenate feed is provided to a first reaction zone. The oxygenate feed comprises oxygenate and C4 olefins. Reference herein to the oxygenate feed is to a single feed comprising oxygenate and C4 olefins or to two or more sub feed each comprising one or more of the compounds of the oxygenate feed, which combined form the oxygenate feed. For instance the oxygenate feed may be provided as sub feed comprising oxygenate and a sub feed comprising C4 olefins.

Preferably, the oxygenate feed provided to step (a) comprises oxygenate and olefins in an oxygenate:olefin molar ratio in the range of from 1000:1 to 1:1, preferably 100:1 to 1:1. More preferably, in a oxygenate:olefin molar ratio in the range of from 20:1 to 1:1, more preferably in the range of 18:1 to 1:1, still more preferably in the range of 15:1 to 1:1, even still more preferably in the range of 12:1 to 1:1. As mentioned above, it is preferred to convert a C4 olefin together with an oxygenate, to obtain a high yield of ethylene and propylene, therefore preferably at least one mole of oxygenate is provided for every mole of C4 olefin.

This first reaction zone may also be referred to as an OTO zone. In the first reaction zone, the oxygenate-comprising feed is contacted with a zeolite-comprising catalyst. The oxygenate-comprising feed is contacted with the catalyst at a temperature in the range of from 350 to 1000° C., preferably of from 450 to 650° C., more preferably of from 530 to 620° C., even more preferably of from 580 to 610° C.; and a pressure in the range of from 0.1 kPa (1 mbar) to 5 MPa (50 bar), preferably of from 100 kPa (1 bar) to 1.5 MPa (15 bar), more preferably of from 100 kPa (1 bar) to 300 kPa (3 bar). Reference herein to pressures is to absolute pressures.

In contact with the zeolite-comprising catalyst at least part of the oxygenates and C4 olefins in the oxygenate feed are converted to olefins. From the first reaction zone a first effluent stream is retrieved comprising at least C2 to C5 olefins. The first effluent stream comprises at least ethylene and/or propylene. In addition, the first effluent stream comprises a C4+ hydrocarbon fraction. Reference herein to a C4+ hydrocarbon is to hydrocarbons comprising 4 or more carbon atoms.

The first effluent stream is subsequently separated into at least:

i. a first product fraction comprising C2 and/or C3 olefins

ii. a second fraction comprising C4 olefins

iii. a third fraction comprising C5 olefins.

The separation of the first effluent stream in the mentioned fractions may be done using any suitable work-up section. The design of the work-up section depends on the exact composition of the olefinic product stream, and may include several separation steps. The design of such a work-up section is well known in the art and does not require further explanation.

The first product fraction may be further treated to retrieve the ethylene and/or propylene at any desired purity required.

The second fraction is a fraction comprising C4 olefins. Although, the second fraction may contain other olefins than C4 olefins, it is preferred that the second fraction comprises in the range of from 80 to 100wt %, more preferably 90 to 100wt %, even more preferably 95 to 100wt % of C4 olefins, based on the weight of the olefins in the second fraction. The olefins having a lower carbon number, i.e. ethylene and propylene are preferably recovered as part of the first product stream, whereas C5 and higher carbon number olefins are preferably retrieved as part of the third fraction and converted in the second reaction zone. While C5 olefins may be converted when provided to the first reaction zone, this is less preferred due to the lower selectivity obtained in the presence of methanol. In the presence of excess methanol, C7 or even higher olefins may be formed due to the overalkylation of the C5 olefins with methanol, which are a precursor for less desired aromatic compounds such as benzene and toluene. It is therefore preferred to minimize the amount of C5 olefins in the second fraction.

Although less desired, the first effluent will typically comprise some aromatic compounds such as benzene, toluene and xylenes. Although it is not the primary aim of the process, xylenes can be seen as a valuable product. Xylenes are amongst others formed in the first reaction zone by the alkylation of benzene and, in particular, toluene with oxygenates such as methanol. Therefore, in a preferred embodiment, a separate fraction comprising aromatics, in particular benzene, toluene and xylenes is separated from the first effluent and at least in part recycled to the first reaction zone as part of the oxygenate feed. Preferably, part or all of the xylenes in the fraction comprising aromatics are withdrawn from the process as a product prior to recycling the fraction comprising aromatic to the first reaction zone.

As mentioned herein above, at least part of the second fraction is recycled back to the first reaction zone. The C4 olefins in the second fraction may react in the first reaction zone with methanol to form further ethylene and propylene. Generally, the effluent of an OTO zone also comprises paraffinic hydrocarbons including C4 paraffins. When these C4 paraffins are recycled to the first reaction zone as part of the second fraction, this may lead to a buildup of a paraffin content in the second fractions, as the C4 paraffins do not, or not to any appreciable extent, react in the first reaction zone under OTO conditions and thus pass through the first reaction zone unconverted. Preferably, therefore at least part of the paraffins in the second fraction is purged from the process. This may be done by withdrawing a certain part of the second fraction, such as between 1 and 5 wt % based on the second fraction, as a purge or a bleed stream.

Another alternative may include the use of extractive distillation with polar liquids to separate the olefins and paraffins in the second fraction. In this way, a paraffin-enriched stream may be purged, while an olefin-enriched stream is recycled.

Preferably, further C4 olefins are provided to the first reaction zone as part of the oxygenate feed, in addition to the C4 olefins from the second fraction.

Oxygenate feed preferably comprises at least 50 wt %, of C4 olefins, more preferably at least 75 wt %, even more preferably at least 90 wt % of C4 olefins, based on the total amount of olefins in the oxygenate feed.

Preferably, at least 70 wt % of any C4 olefin provided to the first reaction zone is, during normal operation, provided as part of the second fraction, preferably at least 90 wt %, based on the C4 olefins provided to the first reaction zone. It will be appreciated that during start-up of the process a significant part of the C4 olefins in the oxygenate feed is provided externally, i.e. not as part of the second fraction. Alternatively, the process is started in the absence of C4 olefins and the C4 olefins are provided as soon as a first effluent comprising C4 olefins is obtained, which C4 olefins may be recycled to form part of the oxygenate feed.

In the process according to the invention, an olefinic feed comprising C5 olefins is provided to a second reaction zone. Reference herein to an olefinic feed is to a feed comprising olefins. This olefinic feed comprises at least part of the third fraction obtained in step(c).

The third fraction comprises C5 olefins, however it may also comprise other olefins in particular C6+ olefins.

Preferably, the third fraction comprises, based on the olefins in the third fraction, at least 90 wt %, preferably at least 95 wt %, of C5+ olefins.

More preferably, the third fraction comprises, based on the olefins in the third fraction, at least 90 wt %, preferably at least 95 wt %, of C5 to C8 olefins.

Even more preferably, the third fraction comprises, based on the olefins in the third fraction, at least 90 wt %, preferably at least 95 wt %, of C5 to C6 olefins.

Still even more preferably, the third fraction comprises, based on the olefins in the third fraction, at least 90 wt %, preferably at least 95 wt %, of C5 olefins.

When the third fraction comprises predominantly C5 olefins, remaining C6+ olefins in the first effluent may be retrieved as a separate fraction from the first effluent stream.

This second reaction zone may also be referred to as an OCP zone. The olefinic feed is contacted in the second reaction zone with a zeolite-comprising catalyst. The feed comprising C5 olefins is contacted with the catalyst at a temperature of in the range of from 500 to 700° C., preferably of from 550 to 650° C., more preferably of from 550 to 620° C., even more preferably of from 580 to 610° C.; and a pressure in the range of from 0.1 kPa (1 mbar) to 5 MPa (50 bar), preferably of from 100 kPa (1 bar) to 1.5 MPa (15 bar), more preferably of from 100 kPa (1 bar) to 300 kPa (3 bar). Reference herein to pressures is to absolute pressures.

In contact with the zeolite-comprising catalyst at least part of the C5 olefins in the olefinic feed is cracked to ethylene and propylene. In addition, C4+ olefins may be formed. From the second reaction zone, a second effluent stream is retrieved comprising at least ethylene and propylene and optionally C4+ olefins. The second effluent may also comprise other hydrocarbons.

As described herein above, the reaction of C5 olefins with oxygenates, in particular when a molar excess of oxygenates is present, may give rise to increased formation of by-product paraffins and aromatics. Therefore, in one embodiment according to the invention the olefinic feedstock does not comprise oxygenates.

However, in contact with the catalyst in the second reaction zone, the C5 olefins in the olefinic feed are cracked to at least ethylene and propylene in an endothermic process, contrary to the process in the first reaction zone, where the reactants are exothermally converted due to the presence of the oxygenate. It has been found that the presence of some oxygenate in the olefinic feed to the second reactor may be beneficial as the exothermic reaction of the oxygenate with the olefins in the olefinic feed may provide part of the energy required to induce the endothermic cracking reactions of the C5 olefins. Therefore, in a further preferred embodiment of the process, the olefinic feed comprises oxygenate. Preferably, the olefinic feed comprises oxygenates and olefins in an oxygenate:olefin molar ratio which is different from the oxygenate:olefin molar ratio of the oxygenate feed, more preferably comprises oxygenates and olefins in an oxygenate:olefin molar ratio which is below the oxygenate:olefin molar ratio of the oxygenate feed. The processes in the first and second reaction zone are different, both requiring different oxygenate:olefin molar ratios. As the reaction in the first reaction zone is primarily an OTO process, it requires oxygenates as a primary reactant, whereas the reaction in the second reaction zone is primarily an C5+ olefin cracking reaction, requiring C5+ olefins as primary reactant. Consequently, it is preferred that the oxygenate:olefin molar ratio of the olefinic feed is below the oxygenate:olefin molar ratio of the oxygenate feed, i.e. the olefinic feed comprises more olefins per mole oxygenate than the oxygenate feed.

In order to reduce by-product formation due to the presence of oxygenate, it is preferred that the molar ratio of oxygenate:olefins is at most 1:1, i.e. at any time the number of moles oxygenate in the olefinic feed is equal to or below the number of moles olefin. However, more preferably the olefinic feed comprises a molar excess of olefins compared to any oxygenate present in the olefinic fed. Preferably, the oxygenate:olefin molar ratio of the olefinic feed is in the range of from 1:1000000 to 1:1, preferably 1:1000000 to below 1:1, more preferably 1:1000000 to 1:2, even more preferably 1:1000000 to 1:10. By maintaining the molar ratio of oxygenate:olefins at 1:1 or below, preferably below 1:1, the formation of C7+olefins by alkylation of the C5 olefins may be reduced compared to a the formation of C7+ olefins in the presence of a molar excess of oxygenates. Any C6 olefins formed are conveniently cracked to predominantly C2, C3 and C4 olefins.

Any oxygenate may be comprised in the olefinic feed, however, preferred oxygenates are tert-amyl ethers. Particularly preferred tert-amyl ethers are tert-amyl methyl ether or tert-amyl ethyl ether, alone or in combination. These tert-alkyl ethers oxygenates have the advantage that they boil in a similar temperature range as the C5 olefins in the olefinic feed. In addition, they provide not only an oxygenate, by the nature of the tert-alkyl ether, they also provide an additional olefin, i.e. the tertiary iso-olefin, which was used to prepare the tert-amyl ether. For purposes of calculating the molar ratio of oxygenate to olefin in the olefinic feed, the olefins provided to the process as part of tert-amyl ether must also be taken into account. The same applies for the oxygenate feed to the first reaction zone should the oxygenate feed comprise one or more tert-alkyl ethers.

It will be appreciated that there may be some oxygenates in the first effluent obtained from the OTO reaction zone, which, although perhaps undesired, may in part end up in the third fraction.

Preferably, further C5 olefins are provided to the second reaction zone as part of the olefinic feed in addition to the C5 olefins from the third fraction. C5 olefins are generally available in refinery waste streams and are, as such, a valuable feedstock for preparing ethylene and propylene.

As mentioned hereinabove, the cracking of C5 olefins can be achieved via a relatively straightforward cracking process that can be operated at high conversion. For instance, in case only C5 olefins are provided to the second reaction zone, an olefin conversion above 80 mol % olefin conversion, preferably above 90 mol %, based on the olefins provided to the second reaction zone may be obtained. In order to attain such high conversions, while maintaining acceptable selectivity towards ethylene and propylene, it is preferred that at least 90 wt %, preferably at least 95 wt %, of the olefins provided to the second reaction zone are C5+ olefins, preferably C5 to C8 olefins, more preferably C5 to C6 olefins, even more preferably C5 olefins.

The second effluent stream may be fractionated to retrieve an ethylene and/or propylene product fraction. Any C4 olefins in the second effluent stream are preferably separated from the second effluent stream and provided to the first reaction zone to be converted to further ethylene and propylene. Any C5 olefins, either unconverted or newly formed in the second reaction zone, in the second effluent stream are preferably separated from the second effluent stream and recycled, at least in part, to the second reaction zone to be converted to further ethylene and propylene. Comparable to the recycle of the second fraction comprising C4, it may be necessary to purge part of the second effluent to remove C5 or C5+ paraffinic hydrocarbons. In case at least part of the C5 olefins in the second effluent stream are recycled, part of the stream that is recycled may be withdrawn from the process as a purge. Similar ways of purging as used for the C4 paraffins may be used.

Preferably, the first and second effluent streams are combined to a combined effluent stream and the combined effluent stream is separated in step (c) to obtain at least:

i. a first product fraction comprising ethylene and/or propylene

ii. a second fraction comprising C4 olefins

iii. a third fraction comprising C5 olefins. Optionally, also a C6+ olefin fraction is obtained and/or a fraction comprising aromatics. In case the first and second effluent are combined to the combined effluent, the teachings and preferences provided herein above for the first effluent and the separation thereof apply mutatis mutandis to the combined effluent.

This has the advantage that both first and second effluent may be separated using the same work-up section. In addition, this provides for an efficient way of providing C4 olefins in the second effluent to the first reaction zone as part of the second fraction. The same applies mutatis mutandis for any C5 olefins in the second effluent stream.

As mentioned above, it may be preferred to provide a tert-amyl ether as part of the olefinic feed. One preferred way of providing these tert-amyl ethers is by allowing tertiary C5 iso-olefins present in the olefinic feed, first effluent, second effluent, combined effluent or third fraction react with an alcohol to form tert-alkyl ethers, preferably C5 iso-olefins present in the third fraction are reacted with an alcohol to form tert-alkyl ethers. Preferably, the alcohol is methanol or ethanol and the tert-amyl ethers are tert-amyl methyl ether or tert-amyl ethyl ether. More preferably, the same alcohol is also comprised in the oxygenate feed. Even more preferably, the alcohol used to form the ether and which is comprised in the oxygenate feed is methanol.

The tertiary iso-olefins may be reacted with the alcohol, such as methanol, in an etherification process. The etherification process may be any suitable etherification process available in the art for etherifying alcohol and tertiary iso-olefins to tert-alkyl ethers. Reference is made to the Handbook of MTBE and Other Gasoline Oxygenates, H. Hamid and M. A. Ali ed., 1^{st} edition, Marcel Dekker, New York, 2004, pages 65 to 223, where several established process and catalyst for preparing tert-amyl ethers such as TAME and TAEE are described. In particular reference is made to chapter 9, pages 203 to 220 of the Handbook of MTBE and Other Gasoline Oxygenates, wherein suitable commercial etherification processes are described. A preferred etherification process is an etherification process wherein the C5 tertiary iso-olefins are converted with methanol to a tert-amyl ether in the presence of a catalyst. Any homogeneous or heterogeneous Brönsted acid may be used to catalyze the etherification reaction. Such catalyst include: sulfuric acid, zeolites, pillared silicates, supported fluorocarbonsulphonic acid polymers and protonated cation-exchange resins catalyst, preferred catalyst are protonated cation-exchange resins catalyst due to the higher catalytic activity and the bound acid sites. A commonly used catalyst is Amberlyst 15.

Preferably, the C5 tertiary iso-olefins are converted with an alcohol to a tert-amyl ether at a temperature in the range of from 30 to 100° C., more preferably 40 to 80° C. Preferably, the C5 iso-olefins are converted with alcohol to a tert-amyl ether at pressures in the range of from 5 to 25 bar, more preferably 6 to 20 bar.

Preferably, the tert-amyl ether is obtained by reacting tertiary isopentenes in the third fraction with an alcohol.

The first and second reaction zones may each be operated as one or more fixed bed reactors, fluidised bed reactors or riser reactors. Preferably, both the first and second reactors are operated as riser reactors. The advantage of the use of a riser reactor is that it allows for very accurate control of the contact time of the several feeds with the catalyst, as riser reactors exhibit a flow of catalyst and reactants through the reactor that approaches plug flow.

The primary operators for controlling the reaction inside the reactor or reaction zone are the gas residence time, the cat/oil ratio and the feed and catalyst inlet temperature. The gas residence time and the cat/oil ratio may be correlated to the earlier mentioned WHSV.

The gas residence time herein refers to the average time it takes for gas at the reactor or reaction zone, inlet to reach the reactor or reaction zone outlet. The gas residence time is also referred to as τ. Preferably, the gas residence time in the second reactor or reaction zone is equal to or below the gas residence time in the first reactor reaction zone. Without wishing to be bound by any particular theory it is believed that when compared on an equal temperature basis, the reaction in the second reaction zone progresses faster than the reaction in the first reaction zone.

The dimensionless cat/oil ratio herein refers to the mass flow rate of catalyst (kg/h) divided by the mass flow rate of the feed (kg/h), wherein the flow rate of the feed is calculated on a CH₂ basis. Preferably, the cat/oil ratio in the second reactor or reaction zone is equal to or below the cat/oil ratio in the first reactor reaction zone. Without wishing to be bound by any particular theory it is believed, that when compared on an equal temperature basis, the reaction in the second reaction zone progresses faster than the reaction in the first reaction zone.

Preferably, the first and second reaction zones are operated under similar temperature conditions. As the reactions taking place in the first riser reaction zone are primarily exothermic, whereas the reactions taking place in the second reaction zone are primarily endothermic, it is preferred that the feed and/or catalyst inlet temperature to the second reaction zone is higher than the temperature of the feed and/or catalyst inlet temperature to the first reaction zone. In order to maintain the temperature in the second reaction zone, heat must be provided to the second reaction zone. This may be done by providing the catalyst or the feed to the second reaction zone at a higher temperature. Preferably, the heat is provided by providing catalyst at a higher temperature, i.e. preferably at a higher temperature compared to the catalyst provided to the first reaction zone. The catalyst can be heated without damaging the catalyst, whereas care must be taken not to induce any decomposition of the feed when preheating the feed to the second reaction zone. Alternatively, the catalyst recirculation rate between the second reaction zone and the catalyst regenerator may be increased to provide more heat to the reaction zone. In the catalyst regenerator, coke may be removed from the catalyst by an oxidation process, which increases the catalyst temperature.

On the contrary, the temperature in the first zone will increase as the reaction progresses and heat of reaction is released. Preferably, at least part of that heat is dissipated by providing the feed or catalyst at a lower temperature to first reaction zone. Preferably, the heat is dissipated by providing the feed at a lower temperature, i.e. preferably at a lower temperature compared to the feed provided to the second reaction zone. The feed can be provided at a lower temperature by reducing the extent of preheating the feed prior to providing the feed to the reaction zone, whereas in case of the catalyst this would require cooling of catalyst that is at a higher temperature, resulting in a loss of energy.

In addition to the oxygenates and olefins, also an amount of diluent is provided to the first reaction zone, either separately, but preferably as part of the oxygenate feed. During the conversion of the oxygenates, steam is produced as a by-product, which serves as an in-situ produced diluent. Optionally, additional steam is added as diluent. The amount of additional diluent that needs to be added depends on the in-situ water make, which in turn depends on the composition of the oxygenate feed. Where the diluent is water or steam, the molar ratio of oxygenate to diluent is between 10:1 and 1:20. Other suitable diluents include inert gases such as nitrogen or methane, but may also include C2-C3 paraffins.

A diluent may also be provided to the second reaction zone together with the olefins. The diluent is provided to the second reaction zone either separately, but preferably as part of the olefinic feed. Preferably, the diluent provided to the second reaction zone is water or steam. Other suitable diluents include inert gases such as nitrogen or methane, but may also include C2-C3 paraffins. Preferably, the diluents provided to the first and second reaction zone are the same, more preferably water or steam.

A variety of OTO processes is known for converting oxygenates to an olefin-containing product, as already referred to above. One such process is described in WO-A 2006/020083. Processes integrating the production of oxygenates from synthesis gas and their conversion to light olefins are described in US20070203380A1 and US20070155999A1.

The first and second zeolite-comprising catalysts suitable for converting the reactants in respectively the first and second reaction zones preferably include zeolite-comprising catalyst compositions. Such zeolite-comprising catalyst compositions typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Zeolites preferably have a molecular framework of one, preferably two or more corner-sharing [TO₄] tetrahedral units, more preferably, two or more [SiO₄], [AlO₄] tetrahedral units.

The first and second zeolite-comprising catalysts suitable for converting the reactants in respectively the first and second reaction zones include those catalyst containing a zeolite of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48.

The above mentioned zeolite-comprising catalysts are suitable for use in both the first and the second reactor zone. Under the appropriate reaction condition, these catalyst may induce the cracking of C5 olefins as well as the conversion of oxygenates alone or together with C4 olefins to ethylene and propylene. These zeolite-comprising catalysts, in particular the ZSM zeolite-comprising catalyst have an advantage over for instance non-zeolite-comprising catalyst such as silicoaluminophosphates like SAPO-34. Although both types of catalyst are suitable to convert oxygenates to olefins, non-zeolite-comprising catalyst are less suitable for converting oxygenates together with olefins such a C4 olefins. The advantage of using zeolites compared to e.g. silicoaluminophosphates becomes even more pronounced when the olefins include iso-olefins such as isobutene.

Preferred catalysts comprise a more-dimensional zeolite, in particular of the MFI type, more in particular ZSM-5, or of the MEL type, such as zeolite ZSM-11. The zeolite having more-dimensional channels has intersecting channels in at least two directions. So, for example, the channel structure is formed of substantially parallel channels in a first direction, and substantially parallel channels in a second direction, wherein channels in the first and second directions intersect.

Intersections with a further channel type are also possible. Preferably the channels in at least one of the directions are 10-membered ring channels. A preferred MFI-type zeolite has a Silica-to-Alumina ratio SAR of at least 60, preferably at least 80.

The zeolite-comprising catalyst may comprise more than one zeolite. In that case it is preferred that the catalyst comprises at least a more-dimensional zeolite, in particular of the MFI type, more in particular ZSM-5, or of the MEL type, such as zeolite ZSM-11, and a one-dimensional zeolite having 10-membered ring channels, such as of the MTT and/or TON type.

The zeolite-comprising catalyst may comprise phosphorus as such or in a compound, i.e. phosphorus other than any phosphorus included in the framework of the zeolite. It is preferred that a catalyst comprising a MEL or MFI-type zeolite additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05 to 10 wt % based on the weight of the formulated catalyst. A particularly preferred catalyst comprises phosphor and MEL or MFI-type zeolites having SAR of in the range of from 60 to 150, more preferably of from 80 to 100. An even more particularly preferred catalyst comprises phosphor and ZSM-5 having SAR of in the range of from 60 to 150, more preferably of from 80 to 100.

It is preferred that zeolites in the hydrogen form are used in the zeolite-comprising catalyst, e.g., HZSM-5, HZSM-11, and HZSM-22, HZSM-23. Preferably at least 50 wt %, more preferably at least 90 wt %, still more preferably at least 95 wt % and most preferably 100 wt % of the total amount of zeolite used is in the hydrogen form. It is well known in the art how to produce such zeolites in the hydrogen form.

The first and second zeolite-comprising catalysts suitable for converting the reactants in respectively the first and second reaction zones may each be separately selected as described herein above. However, it is a particular advantage of the present invention the first and second zeolite-comprising catalyst may be the same. By using the same catalyst in the first and second reaction zone it is possible to use for instance a single catalyst storage or catalyst regeneration facility. More importantly, by using the same catalyst in both the first and second zeolite-comprising catalyst there is no risk of contamination of the first zeolite-comprising catalyst inventory in the first reaction zone with the second zeolite-comprising catalyst or vice versa. Such contamination could for instance occur when small amounts of catalyst are retained in effluent of the first or second reaction zone, which catalyst may end up in the second or third fraction.

Preferably, the zeolite-comprising catalyst containing phosphorus has been prepared by a process which includes at least the following steps:

v) preparing an aqueous slurry comprising a zeolite, clay material and binder;

vv) spraydrying the aqueous slurry to obtain zeolite-comprising catalyst particles;

vvv) treating the spraydried zeolite-comprising catalyst particles with phosphoric acid to introduce phosphorus compounds on the spraydried and zeolite-comprising catalyst particles; and

vvvv) calcining the spraydried zeolite- and phosphorus-comprising catalyst particles.

Preferably, the residence time of the reactants in the first reaction zone, also referred to as τ, is in the range of from 1 to 10 seconds, more preferably of from 3 to 6 seconds, even more preferably of from 3.5 to 4.5 seconds.

Preferably, the cat/oil ratio i.e. on a CH₂ basis for hydrocarbons including oxygenates, in the first reaction zone is in the range of from 1 to 100, more preferably of from of from 1 to 50, even more preferably 5 to 25.

It is preferable to control the severity of the process in the first reaction zone. When the process is operated at a too high severity, side reactions increase as well as by-product formation at the cost of ethylene and propylene selectivity. In case, the severity is too low, the process is operated inefficient and sub optimal conversions are obtained. The severity of the process is influenced by several reaction and operation conditions, however a suitable measure for the severity of the process in the first reaction zone is the C5 olefin content in the effluent of the first reaction zone. A higher C5 olefin content indicates lower severity and vice versa. Preferably, the reaction conditions in the first reaction zone are chosen such that the first effluent stream comprises in the range of from 7.5 to 40 wt % of C5 olefins, based on the olefins in the first effluent, preferably 12.5 to 30 wt % of C5 olefins. The C5 content in the effluent of the first reaction zone depends on the severity of the reaction and the C5 content in the effluent, and thus also the severity of the reaction, may be controlled by changing one of more of the reaction conditions. One such condition is the temperature in the first reaction zone. As the temperature is reduced the C5 olefin content of the first effluent may increase and vice versa where the aim is to reduce the C5 olefin content of the first effluent. Furthermore, reducing the residence time of the reactants in the reactor may also increase the C5 olefin content in the first effluent and vice versa where the aim is to reduce the C5 olefin content of the first effluent. Alternatively, reducing the cat/oil ratio may also increase the C5 olefin content in the first effluent and vice versa. One other way of increasing the C5 content in the first effluent is by using a less active catalyst. This may be achieved by either operating the process with a catalyst having a higher average coke load or by reducing the catalyst refresh rate, i.e. the rate of replacement of spent catalyst by fresh catalyst. Where the aim is to reduce the C5 olefin content of the first effluent, the catalyst activity may be increased by the reverse of these measures. It will be appreciated that any combination of the above described measures may influence the C5 olefin content of the first effluent. It is well within the skills of the person skilled in the art to select the most appropriate measure. Preferably, the C5 olefin content of the first effluent is controlled by adjusting the residence time and/or the cat/oil ratio, as these are adjusted most conveniently. As mentioned above, in case the C5 content in the first effluent is higher than preferred, the above described measures may be used mutatis mutandis, i.e. increased temperature, residence time, cat/oil ratio and catalyst activity. Two or more of the above described measures may be used, in addition to others, to control the C5 content in the first effluent. The C5 content in the first effluent is conveniently analyzed using any suitable means of analyzing the hydrocarbon content in a process stream. Particular suitable means of analyzing the C5 content in the first effluent include gas chromatography and near infrared spectrometry.

Preferably, the reaction conditions in the first zone are chosen such that the oxygenate conversion is in the range of from 90 to 100%, based on the oxygenates provided to the first reaction zone, preferably 95 to 100%.

Preferably, the residence time of the reactants in the second reaction zone, also referred to as τ, is in the range of from 1 to 10 seconds, more preferably of from 3 to 6 seconds, even more preferably of from 3.5 to 4.5 seconds.

Preferably, the cat/oil ratio in the second reaction zone is in the range of from 1 to 100, more preferably of from 1 to 50, even more preferably of from 5 to 25.

Typically, the catalyst deactivates in the course of the process, amongst other things due to deposition of coke on the catalyst. Conventional catalyst regeneration techniques can be employed to remove the coke. It is not necessary to remove all the coke from the catalyst as it is believed that a small amount of residual coke may enhance the catalyst performance and additionally, it is believed that complete removal of the coke may also lead to degradation of the zeolite. This applies to both the catalyst used in step (b) of the process as well as the catalyst in the step (f) of the process.

The catalyst particles used in the process of the present invention can have any shape known to the skilled person to be suitable for this purpose, for it can be present in the form of spray dried catalyst particles, spheres, tablets, rings, extrudates, etc. Extruded catalysts can be applied in various shapes, such as, cylinders and trilobes. If desired, spent oxygenate conversion catalyst can be regenerated and recycled to the process of the invention. Spray-dried particles allowing use in a fluidized bed or riser reactor system are preferred. Spherical particles are normally obtained by spray drying. Preferably the average particle size is in the range of 1-200 μm, preferably 50-100 μm.

### EXAMPLES

The invention is illustrated by the following non-limiting examples.

### Catalyst Preparation

A ZSM-5 zeolite powder with a SAR of 80 was first calcined at 550° C. The calcined zeolite was added to an aqueous solution and subsequently the slurry was milled. Kaolin clay and a silica sol were added and the resulting mixture was spray dried wherein the weight-based average particle size was between 70-90 μm. The spray dried catalysts were exposed to ion-exchange using an ammonium nitrate solution. Phosphorus was deposited on the catalyst by means of impregnation using an acidic solutions containing phosphoric acid (H₃PO₄). The concentration of the solution was adjusted such to impregnate 1.5 wt % of phosphorus on the catalyst. After impregnation the catalysts were dried at 140° C. and were calcined at 550° C. for 2 hours.

The phosphorus loading on the final catalysts is given based on the weight percentage of the elemental phosphorus in any phosphor species, based on the total weight of the formulated catalyst.

To test the catalyst formulations for catalytic performance in the microflow experiments, the catalysts were pressed into tablets and the tablets were broken into pieces and sieved (60-80 mesh). In the fluidized-bed reactor experiments the catalyst formulation was used as prepared.

### Example 1

Methanol, 1-butene and a mixture of 1-butene and methanol were reacted over the catalyst to determine the selectivity of the reaction toward in particular ethylene and propylene. For these experiments, the catalysts were pressed into tablets and the tablets were broken into pieces and sieved. A sieve fraction of 60-80 mesh of the crushed catalyst was used.

The reaction was performed using a quartz reactor tube of 1.8 mm internal diameter. The catalyst was first heated in nitrogen to the reaction temperature. In a first experiment (1a), 6 vol % of methanol balanced in N₂ was passed over the catalyst at atmospheric pressure (1 bar and 525° C.). In a following experiment (1b), 3 vol % of butene-1 balanced in N₂ was passed over the catalyst at atmospheric pressure (1 bar and 525° C.). In a third experiment (1c) a mixture consisting of 3 vol % butene-1 and 6% vol % methanol balanced in N₂ was passed over the catalyst at atmospheric pressure (1 bar and 525° C.)

The Gas Hourly Space Velocity (GHSV) is determined by the total gas flow over the zeolite weight per unit time (cm³.gzeolite⁻¹.h⁻¹). The gas hourly space velocity used in the experiments was 19000 (cm³.gzeolite⁻¹.h⁻¹).

In a similar experiment, 25 vol % of 1-pentene balanced in N₂ was passed over the catalyst at atmospheric pressure (1 bar) at two different temperatures 525° C. (experiment 1d) and 600° C. (experiment le). The GHSV used in experiments (1d) and (1e) was 7800 (cm³.gzeolite⁻¹.h⁻¹) .

The effluent from the reactor was analyzed by gas chromatography (GC) to determine the product composition. The composition has been calculated on a weight basis of all hydrocarbons analyzed. The composition has been defined by the division of the mass of specific product by the sum of the masses of all products. The effluent from the reactor obtained at several reactor temperatures was analyzed. The results are shown in Table 1.

Selectivity is defined as the ratio between the ethylene and propylene yield and the sum of the yields of olefins and the by-product. The yield is defined as the ratio of a product formed to the fraction of feed converted.

TABLE 1

| | Feed | | | Product | | | | | | | | | |
|---|---|---|---|---|---|---|---|---|---|---|---|---|---|
| | (diluent free | | | (diluent free basis) | | | | | | | | Selectivity | |
| | basis) | | | | | | | Other by- | Conversion | | | to | Ratio |
| | MeOH | C₄⁼ | C₅⁼ | C₂⁼ | C₃⁼ | C₄⁼ | C₅₊⁼ | products | MeOH | C₄⁼ | C₅⁼ | C₂⁼ + C₃⁼ | C₂⁼/C₃⁼ |
| Exp. | [wt %] | [wt %] | [wt %] | [wt %] | [wt %] | [wt %] | [wt %] | [wt %] | [%] | [%] | [%] | [%] | [—] |
| 1a | 100 | 0 | 0 | 14.6 | 50.8 | 25.6 | 5.7 | 3.3 | 100 | n.a. | n.a. | 95.2 | 0.29 |
| 1b | 0 | 100 | 0 | 8.00 | 23.7 | 62.0 | 2.7 | 3.6 | n.a. | 38.0 | n.a. | 89.8 | 0.34 |
| 1c | 53.3 | 46.7 | 0 | 16.7 | 50.0 | 22.5 | 8.2 | 2.6 | 100 | 52.0 | n.a. | 96.3 | 0.33 |
| 1d | 0 | 0 | 100 | 16.4 | 37.5 | 23.7 | 13.5 | 8.9 | n.a. | n.a. | 90.2 | 85.8 | 0.44 |
| (525° C.) | | | | | | | | | | | | | |
| 1e | 0 | 0 | 100 | 28.1 | 40.6 | 17.4 | 7.6 | 6.4 | n.a. | n.a. | 95.3 | 91.5 | 0.694 |
| (600° C.) | | | | | | | | | | | | | |
| 1f* | | 50 | 50 | 12.2 | 30.6 | 42.8 | 8.1 | 6.3 | n.a. | 14.3 | 83.8 | 87.2 | 0.40 |

*Calculated as an 1:1 average example (1b) and (1d)

When comparing the result form experiments (1a), (1b) and (1c), it can be seen that the conversion of the butenes can be increased by more than 35%, by adding methanol to the butenes when contacting the butenes with the catalyst. Methanol alone can be converted to ethylene and propylene using the prepared catalyst. The addition of butenes to the methanol does not negatively influence the conversion of methanol, actually, by providing a mixture of methanol and butenes, a higher butene conversion is obtained, while the methanol conversion remains 100%. The mixture of methanol and butenes results in a higher ethylene to propylene ratio compared to butenes alone, while being comparable to that of methanol alone. In addition, the yield towards ethylene and propylene is increased compared to either butene alone or methanol alone, while at the same time byproduct formation is reduced. More C5+ olefins are produced, however, these are conveniently converted in the second reaction zone. As can be seen from experiment (1d), pentene can be converted into ethylene and propylene in contact with the prepared catalyst. Where only approximately 38 wt % of the butenes were converted when contacted with the catalyst (1b), over 85 wt % of the pentenes are converted in a single pass. An additional benefit is obtained as the ratio of ethylene to propylene is approximately 0.44, which is significantly above the ratio of ethylene to propylene obtained for converting butenes alone.

In calculated experiment (1f), an effluent composition has provided being the calculated average of the effluent composition of experiments (1b) and (1d). Experiment (1f) models the effluent that is obtained when contacting a feed comprising a mixture of butenes and pentenes with the prepared catalyst, on the basis that butenes and pentenes crack independently from each other.

Where a combination of experiments 1c (step (b) according to the invention) and 1d or le (step (e) according to the invention) represents the process according to the invention, a combination of experiments (1a) and (1f) represents a process according to the prior art, i.e. MTO followed by OCP. It will be clear from experiments (1a) and (1f), that the addition of butenes to a pentene feed negatively influences composition of the effluent in terms of selectively toward ethylene and propylene, total feed conversion and ethylene over propylene ratio compared to a combination of experiments (1c) and (1d) or (1e), where the butene is converted with methanol, resulting in an improved conversion of both methanol and butenes, while the pentenes are converted separately. The obtained overall product is improved in terms of selectively toward ethylene and propylene, total feed conversion and ethylene over propylene ratio.

In addition to the above, experiment (1e) shows the benefit of cracking the pentenes a higher temperature. As can be seen from Table 1, the selectively toward ethylene and propylene, the conversion of the pentenes as well as the ratio of ethylene to propylene are increased. By converting the pentenes separately from the butenes the temperature of the reaction in the second reaction zone can be increased, without increasing byproduct formation caused by butenes in the feed to the second reaction zone.

### Example 2

A dimethyl ether (DME) and butane-1 feed was converted over the prepared catalyst in a fluidized bed reactor having an internal volume of 100 ml. The reactor was placed inside an oven. A feed of 27 vol % DME, 3 vol % butane-1 balanced with dilution steam was passed through the reactor and contacted with the fluidized catalyst. The pressure in the reactor was 1.8 bara and the internal reaction temperature was controlled to remain in between 600 and 610° C. Every 30 minutes, the reaction was halted and the catalyst was regenerated with air for 10 minutes at 600° C. The reaction and regeneration sequence was carried out 10 times.

The product gas for analysis was sampled before terminating the feed and was analyzed in a Gas Chromatography (GC). The product compositions were calculated as a carbon based weight percentage of the hydrocarbons analyzed. The ethylene and propylene yield is defined as the ratio of the ethylene and propylene formed to the fraction of feed converted. Selectivity is defined as the ratio between the ethylene and propylene yield and the sum of the yields of olefins and the by-product. The results are shown in Table 2.

### Example 3

Using the reactor set-up and experimental procedure as used in Example 2, a pentene-1 feed was converted over the prepared catalyst. The feed comprised 20.4 vol % pentene-1 balanced in dilution steam. The results are shown in Table 2.

### Example 4

Using the reactor set-up and experimental procedure as used in Example 2, a dimethyl ether (DME), butane-1 and pentene-1 feed was converted over the prepared catalyst. The feed comprised 27 vol % DME, 1.5 vol % butene, 1.5 vol % pentene-1 balanced with dilution steam. The results are shown in Table 2.

TABLE 2

| | | Product (diluent free basis) | | | | | | | |
|---|---|---|---|---|---|---|---|---|---|
| | | Paraffins + | | | | | | Selectivity | |
| | | Cyclics + | | | | | | to | Yield |
| | WHSV | Aromatics | C₂⁼ | C₃⁼ | C₄⁼ | C₅₊⁼ | C₆₊⁼ | C₂⁼ + C₃⁼ | C₂⁼ + C₃⁼ |
| Exp. | [1/h] | [wt %] | [wt %] | [wt %] | [wt %] | [wt %] | [wt %] | [%] | [%] |
| 2 | 12 | 10.5 | 15.6 | 36.0 | 20.9 | 10.9 | 3.4 | 77.5 | 63.3 |
| 3 | 10 | 14.7 | 24.4 | 34.5 | 17.9 | 4.5 | 2.9 | 79.0 | 61.7 |
| 4 | 9.7 | 21.4 | 19.6 | 34.0 | 16.9 | 4.5 | 1.8 | 69.1 | 67.4 |

TABLE 3

| | | Product (diluent free basis) | | | | | | | |
|---|---|---|---|---|---|---|---|---|---|
| | | Paraffins + | | | | | | Selectivity | |
| | | Cyclics + | | | | | | to | Yield |
| | WHSV | Aromatics | C₂⁼ | C₃⁼ | C₄⁼ | C₅₊⁼ | C₆₊⁼ | C₂⁼ + C₃⁼ | C₂⁼ + C₃⁼ |
| Exp. | [1/h] | [wt %] | [wt %] | [wt %] | [wt %] | [wt %] | [wt %] | [%] | [%] |
| 2 | 12 | 10.5 | 15.6 | 36.0 | 20.9 | 10.9 | 3.4 | 77.5 | 63.3 |
| 5 | 2.4 | 27.7 | 19.1 | 29.7 | 16.1 | 4.8 | 1.5 | 62.9 | 58.2 |

When a mixture of DME and butene is converted to ethylene/propylene with 63.3% yield (experiment 2) and pentenes from the product effluent of experiment 2 are catalytically cracked separately (experiment 3) with 79% selectivity, the combined yield to ethylene/propylene of the process according to the invention increases from 63.3% to 72%. On the other hand, by converting pentenes through recycling pentenes in the effluent of an MTO process together with the butenes in the effluent, while feeding additional DME, (experiment 4) a yield of 67.4% was obtained. This clearly shows the improved performance by cracking the C5 olefins separately rather recycling the pentenes with the butenes.

### Example 5

The experiment of Example 2 was repeated to show the effect of operating the process at a higher severity, i.e. a severity as measured by the C5 olefin content in the effluent. The severity of the process was increased by reducing the weight hourly space velocity (WHSV) to 2.4 h⁻¹ compared to a WHSV of 12 in Example 2. By lowering the WHSV, the severity of the process is increased, which effect is also obtained when increasing the cat/oil ratio, residence time or both.

The results are provided in Table 3.

By lowering the WHSV to 2.4 h⁻¹, the severity of the process is increased and the C5 content in the effluent drops to 6.7 wt % on an olefin basis, compared to 12.5 wt % for the process operated at a WHSV of 12h⁻¹. By operating the process under the desired severity, i.e. a severity, whereby the C5 olefin content in the effluent of the first zone ranges of from 7.5 to 40 wt %, a higher ethylene and propylene yield is obtained as well as a higher selectivity towards ethylene and propylene.

1. A process for preparing ethylene and/or propylene, comprising the steps of:
a) providing an oxygenate feed comprising oxygenate and C4 olefins to a first reaction zone;
b) contacting in the first reaction zone the oxygenate feed with a first zeolite-comprising catalyst at a temperature in the range of from 350 to 1000° C. and retrieving from the first reaction zone a first effluent stream comprising at least C2 to C5 olefins;
c) separating the first effluent stream into at least:
i. a first product stream comprising C2 and/or C3 olefins;
ii. a second fraction comprising C4 olefins; and
iii. a third fraction comprising C5 olefins;
d) recycling at least part of the second fraction to the first reaction zone as part of the oxygenate feed;
e) providing an olefinic feed comprising C5 olefins to a second reaction zone, wherein the olefinic feed comprises at least part of the third fraction; and
f) contacting in the second reaction zone the olefinic feed with a second zeolite-comprising catalyst at a temperature in the range of from 500 to 700° C. and retrieving from the second reaction zone a second effluent stream comprising at least C2 to C3 olefins.

2. A process according to claim 1, wherein in step (a) the oxygenate feed comprises oxygenate and olefins in an oxygenate:olefin molar ratio in the range of from 1000:1.

3. A process according to claim 1, wherein the olefinic feed comprises oxygenates.

4. A process according to claim 3, wherein the oxygenate:olefin molar ratio of the olefinic feed is in the range of from 1:1000000 to 1:1.

5. A process according to claim 3, wherein the oxygenate is a tert-amyl methyl ether and/or tert-amyl ethyl ether.

6. A process according to claim 1, wherein the olefinic feed does not contain oxygenate.

7. A process according to claim 1, wherein the first and second effluent streams are combined to a combined effluent stream and the combined effluent stream is separated in step (c) to obtain at least the first, second and third fraction.

8. A process according to claim 1, wherein further C5 olefins are provided to the second reaction zone as part of the olefinic feed in addition to the C5 olefins from the third fraction.

9. A process according to claim 1, wherein, at least 90 wt.

10. A process according to claim 1, wherein first zeolite-comprising catalyst and the second zeolite-comprising catalyst are the same zeolite-comprising catalyst.

11. A process according to claim 1, wherein the first and second zeolite-comprising catalyst are a zeolite-comprising catalyst containing phosphorus.

12. A process according to claim 11, wherein the zeolite-comprising catalyst containing phosphorus has been prepared by a process which includes at least the following steps:
v) preparing an aqueous slurry comprising a zeolite, clay material and binder;
vi) spraydrying the aqueous slurry to obtain zeolite-comprising catalyst particles;
vii) treating the spraydried zeolite-comprising catalyst particles with phosphoric acid to introduce phosphorus compounds on the spraydried and zeolite-comprising catalyst particles; and
viii) calcining the spraydried zeolite- and phosphorus-comprising catalyst particles.

13. A process according to claim 11, wherein the zeolite-comprising catalyst containing phosphorus contains phosphorus in an elemental amount in the range of from 0.05-10 wt % based on the weight of the catalyst.

14. A process according to claim 1, wherein the first and second zeolite-comprising catalysts comprise at least one zeolite selected from MFI, MEL, TON and MTT type zeolites.

15. A process according to claim 11, wherein the oxygenate feedstock comprises at least methanol.
